(19) United States
(12) Patent Application Publication
(10) Pub. No.: US 2009/0124577 A1
(43) Pub. Date: May 14, 2009

(54) Intermediate Compounds for the Preparation of an Angiotensin II Receptor Antagonist

(75) Inventors: Llorenc Rafecas Jane, Barcelona (ES); Antoni Riera Escale, Barcelona (ES); Marta Ecija Queralt, Barcelona (ES); Albert Moyano Baldoire, Barcelona (ES); Alex Comely, Barcelona (ES); Irene Casalprim Castella, Barcelona (ES)

Correspondence Address: BERENBAUM, WEINSHIENK & EASON, P.C, 370 17TH STREET, SUITE 4800, DENVER, CO, 80202, US

(73) Assignee: Enantia SI

(21) Appl. No.: 11/722,640

(22) PCT Filed: Dec. 22, 2005
(86) PCT No.: PCT/EP05/57104

§ 371 Date: Jun. 22, 2007

(30) Foreign Application Priority Data
Dec. 22, 2004 (EP) 04106863.6

(51) Int. Cl. A61K 31/69; C07F 5/04

(52) U.S. Cl. 514/64; 558/287

(57) ABSTRACT

It comprises new substituted 4-valinylmethylphenyl boronic acids of formula (II) and their derivatives and also its preparation process. It also comprises a preparation process of Valsartan (I) from such intermediates. The process comprises the reaction of the new 4-valinylmethylphenyl boronic compounds with a (halophenyl)tetrazole compound which proceeds with high yields. The process is particularly advantageous in its practical industrial realization because it avoids the use of azide derivatives and also the use of expensive biphenyl intermediates.

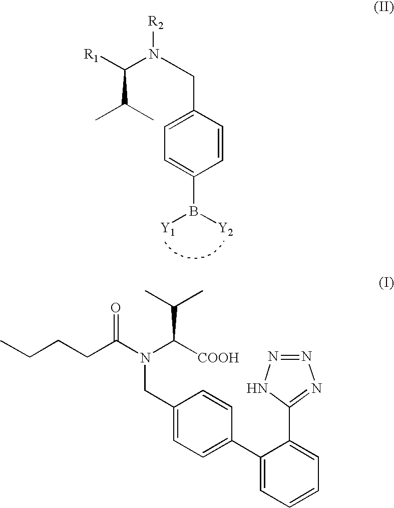

The present invention relates to new 4-valinylmethylphenyl boronic acids and their derivatives, which are intermediates useful for the preparation of Valsartan. It relates also to their preparation process, as well as to a process for the preparation of Valsartan from such intermediates.

### BACKGROUND ART

Valsartan is the generic name of the N-pentanoyl-N-{[2′-(1H-tetrazol-5-yl)-1,1′-biphenyl-4-yl]methyl}-L-valine, having the formula (I).

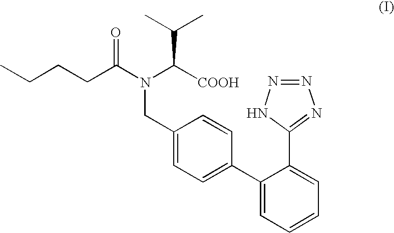

Valsartan is an Angiotensin II (A-II) receptor antagonist known since the publication of the European patent EP443983-A. In this document two preparation processes of Valsartan are described. Both processes involve a coupling between the biphenyl moiety and the valine moiety which is carried out by reductive amination, and subsequently N-acylation to introduce the acyl moiety.

Valsartan could also be prepared by a process which involves the coupling of both phenyl moieties. In EP994881-A a preparation process of 2-substituted-1-(tetrazol-5-yl)benzenes by coupling a suitable bromophenyl derivative with an ortho-methalated (tetrazol-5-yl)benzene is described. According to this patent application, such compounds are useful intermediates for the preparation of several Angiotensin II antagonists.

Thus, from what is known in the art it is derived that the provision of an efficient alternative preparation process of Valsartan from new intermediates would be of great interest in industry.

### SUMMARY

Described here is a new preparation process of Valsartan from new intermediate boron compounds, which can proceed with high yield and high purity.

Thus, according to an aspect hereof, there is provided a compound of formula (II),

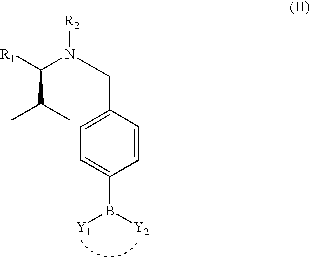

where: Y₁ and Y₂ are each independently selected from the group consisting of hydroxy, (C₁-C₄)-alcoxy and phenoxy, the latter optionally substituted by a (C₁-C₄)-alcoxy, (C₁-C₄)-alkyl or a halogen group; or alternatively Y₁ and Y₂ can be taken together with a boron atom to form a cyclic structure selected from the following ones,

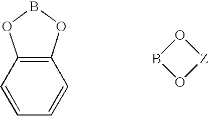

wherein Z is selected from the group consisting of (CH₂)ₙ, (CH₂)ᵣCRᵤRᵥ(CH₂)ₛ and CRᵤRᵥ(CH₂)ₜCRᵤRᵥ; n is an integer from 2 to 4; r and s are integers from 0 to 4 with the condition that r and s are not both 0; t is an integer from 0 to 1, and Rᵤ and Rᵥ are each independently selected from the group consisting of H, (C₁-C₄)-alkyl, phenyl and mono- or di- substituted phenyl, the substituents being halogen, (C₁-C₄)-alkyl and (C₁-C₄)-alkoxy;
R₁ represents a group which may be converted into a carboxy group; and R₂ is a radical selected from H and pentanoyl.

Unless otherwise indicated, the term “carboxy” is used to refer to the radical —COO— as free acid (i.e —COOH) or in salt form.

Another aspect hereof relates to a preparation process of a compound of formula (II) as defined above, which comprises condensing a compound of formula (III) with a compound of formula (IV) or a salt thereof, followed by reducing the condensation product,

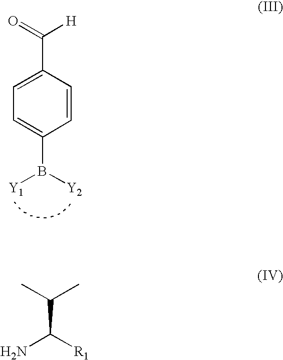

wherein in formulae (III) and (IV), R₁, Y₁ and Y₂ are groups as defined above, or alternatively, Y₁ and Y₂ are intermediate forms thereof which can be transformed to such Y₁ and Y₂ groups; and thereafter, as necessary, transforming said intermediate forms of Y₁ and Y₂ groups to Y₁ and Y₂ groups as previously defined, and optionally submitting the compound obtained to an acylation reaction using a pentanoyl halide to give a compound of formula (II) with R₂=pentanoyl.

In still another aspect hereof there is provided a preparation process of Valsartan of formula (I) or a pharmaceutical salt thereof,

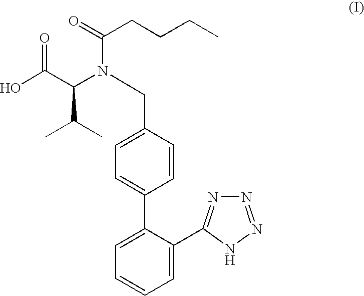

which comprises: a) a coupling reaction of a compound of formula (II) as defined above,

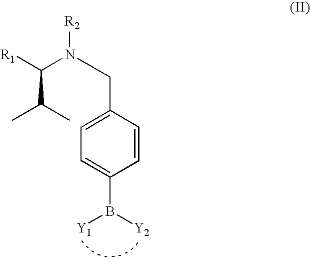

with a compound of formula (V), wherein Y is a leaving group and P′ is H or a protective group P;

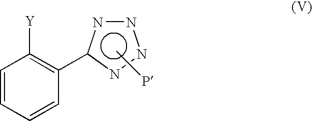

in an appropriate solvent system and in the presence of a metallic compound and a base to give a compound of formula (I′),

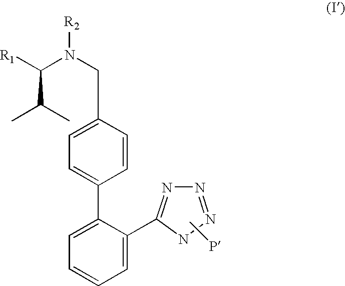

where R₁, R₂ have the same meaning as in formula (II) and P′ is H or a protective group P; b) as necessary, submitting the compound obtained to a deprotection reaction to remove the protective group P′ and/or, as necessary, to an acylation reaction with a pentanoyl halide to introduce the pentanoyl moiety; and thereafter converting the compound of step a) or the compound obtained in said step b) into the free acid form of Valsartan or a salt thereof, by a hydrolysis, thermolysis, acidolysis or hydrogenolysis reaction; or alternatively, firstly submitting the compound of step a) to a hydrolysis, thermolysis, acidolysis or hydrogenolysis reaction to yield the free acid form of valsartan or a salt thereof, or of an intermediate form of valsartan, and thereafter as necessary, submitting the compound obtained to a deprotection reaction to remove the protective group P′ and/or, as necessary, to an acylation reaction with a pentanoyl halide to introduce the pentanoyl moiety; and c) if desired, converting the resulting free acid form or a salt of Valsartan obtained in the step b) into a salt thereof, or converting a resulting salt of Valsartan into the free acid form of Valsartan, or converting a resulting salt of Valsartan into a different salt.

### DETAILED DESCRIPTION

Preferred compounds of formula (II) are those where R₁ is COOR′, R′ being a radical selected from (C₁-C₆)-alkyl such as methyl or ethyl; substituted methyl such as methoxy methyl; 2-substituted ethyl such as tert-butyl or 2,2,2-trichloroethyl; 2,6-dialkylphenyl such as 2,6-dimethylphenyl; benzyl; substituted benzyl such as p-methoxybenzyl, benzhydryl or trityl; and silyl.

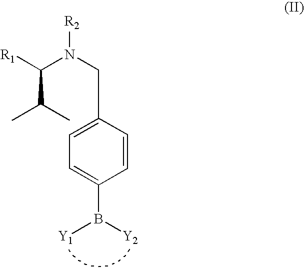

In a more preferred implementation, compounds of formula (II) are those where R₁ is COOR′ with R′=methyl. Also in a more preferred implementation, compounds of formula (II) are those where R₁ is COOR′ with R′=tert-butyl, and also in a more preferred implementation, compounds of formula (II) are those where R₁ is COOR′ with R′=benzyl.

In another preferred embodiment, compounds of formula (II) are those where Y₁ and Y₂ are independently selected from hydroxy, methoxy, ethoxy and phenoxy, or alternatively, Y₁ and Y₂ together with a boron atom form a cyclic structure, wherein Z is selected from the group consisting of (CH₂)ᵣCRᵤRᵥ(CH₂)ₛ and CRᵤRᵥ(CH₂)ₜCRᵤRᵥ; r and s are integers from 0 to 4 with the condition that r and s are not both 0; t is an integer from 0 to 1 and Rᵤ and Rᵥ are each independently selected from methyl and phenyl. In another more preferred implementation compounds of formula (II) are those where Y₁ and Y₂ are hydroxy. In an also more preferred implementation, compounds of formula (II) are those where Y₁ and Y₂ together with a boron atom form a cyclic structure, wherein Z is CH₂C(CH₃)₂CH₂. In an also more preferred implementation, compounds of formula (II), are those where Y₁ and Y₂ together with a boron atom form a cyclic structure, where Z is C(CH₃)₂C(CH₃)₂.

The most preferred compounds of formula (II) are those of the following list:

methyl N-(4-(5,5-dimethyl-[1,3,2]dioxaborinan-2-yl)phenyl-4-yl-methyl)-L-valinate;
methyl N-(4-(5,5-dimethyl-[1,3,2]dioxaborinan-2-yl)phenyl-4-yl-methyl)-N-pentanoyl-L-valinate;
methyl N-(4-(4,4,5,5-tetramethyl-[1,3,2]dioxaborolan-2-yl)phenyl-4-yl-methyl)-L-valinate;
methyl N-(4-(4,4,5,5-tetramethyl-[1,3,2]dioxaborolan-2-yl)phenyl-4-yl-methyl)-N-pentanoyl-L-valinate;
benzyl N-(4-(5,5-dimethyl-[1,3,2]dioxaborinan-2-yl)phenyl-4-yl-methyl)-L-valinate;
benzyl N-(4-(5,5-dimethyl-[1,3,2]dioxaborinan-2-yl)phenyl-4-yl-methyl)-N-pentanoyl-L-valinate;
tert-butyl N-(4-(5,5-dimethyl-[1,3,2]dioxaborinan-2-yl)phenyl-4-yl-methyl)-L-valinate;
tert-butyl N-(4-(5,5-dimethyl-[1,3,2]dioxaborinan-2-yl)phenyl-4-yl-methyl)-N-pentanoyl-L-valinate;
4-[(1-(S)-methoxycarbonyl-2-methyl-propylamino)-methyl]phenylboronic acid; and
4-{[(1-(S)-methoxycarbonyl-2-methyl-propyl)-pentanoyl-amino]-methyl}phenylboronic acid.

As previously described, compounds of formula (II) are prepared by a process, which comprises condensing a compound of formula (III) with a compound of formula (IV) or a salt thereof, by removing water; and reducing the condensation product. This two-step reaction is known as reductive amination.

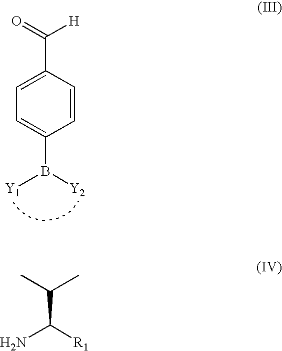

In formulae (III) and (IV), R₁, Y₁ and Y₂ are groups as previously defined.

The elimination of water can be performed by azeotropic removal or using a water scavenger. A preferable water scavenger to carry out the process hereof is MgSO₄. However, other suitable water scavengers can be used such other inorganic sulphates, anhydrides of organic acid, anhydrides of inorganic acid, aluminosilicates such as molecular sieves, zeolites, and inorganic salts.

Preferably, the condensation reaction is carried out in the presence of a base and an appropriate solvent. More preferably, the base is a tertiary amine such as triethylamine, diisopropylethylamine, N-methylmorpholine and pyridine. The most preferred base is triethylamine and examples of suitable solvents are tetrahydrofuran or toluene. Preferably, the reaction is carried out at room temperature.

The reduction can be carried out without isolation of the imine intermediate obtained in the condensating reaction. Preferably, the reduction of the condensation product is carried out with a reducing agent selected from the group consisting of an alkali metal borohydride such as sodium borohydride, an alkali metal cyanoborohydride such as sodium cyanoborohydride or lithium cyanoborohydride, an alkali metal tri-(C₁-C₇)alkoxy borohydride such as sodium tri-methoxyethoxy-borohydride; a tetra-C₁-C₇-alkylammonium-(cyano)borohydride such as tetrabutylammonium borohydride or tetrabutylammonium cyanoborohydride, in the presence of a suitable solvent. A suitable catalyst for the reductive amination with hydrogen or a hydrogen donor is, for example, nickel, such as Nickel Raney, and noble metals or their derivatives such as palladium, platinum or platinum dioxide. Preferably, the reaction is carried out at room temperature.

When Y₁ and Y₂ are intermediate forms of Y₁ and Y₂, the preparation process includes, as necessary, transforming said intermediate forms of the Y₁ and Y₂ groups to Y₁ and Y₂ groups as previously defined.

Likewise, when in formula (II) R₂ is pentanoyl, the process comprises an additional step comprising an acylation reaction with a pentanoyl halide. Preferably, the acylation is carried out with pentanoyl chloride in the presence of a base and a suitable solvent. Preferably, the base is a tertiary amine such as triethylamine, diisopropylethylamine, N-metylmorpholine and pyridine, and more preferably, the tertiary amine is triethylamine. Examples of suitable solvents are dichloromethane, toluene, dioxane or a mixture of tetrahydrofuran with water. Generally, the reaction is between room and reflux temperature. Preferably, it is carried out at room temperature.

Compounds of formula (III) may be obtained from 4-formylphenylboronic acid by methods known in the art. For instance, Example 1 illustrates the preparation of 4-(5,5-dimethyl-[1,3,2]dioxaborinan-2-yl)-benzaldehyde by reaction of 4-formylphenylboronic acid with 2,2-dimethyl-1,3-propanediol.

It is possible to carry out two or more of the steps of the process in one pot, as illustrated in Examples.

Compounds of formula (II) with R₁, R₂, Y₁ and Y₂ as defined above are useful intermediates for the preparation of Valsartan of formula (I) or a pharmaceutical salt thereof.

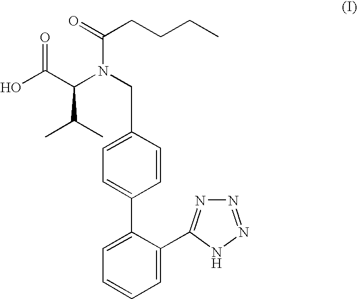

As previously described, the preparation process of Valsartan comprises a coupling reaction of the new compound of formula (II) with a (halophenyl)tetrazole compound of formula (V),

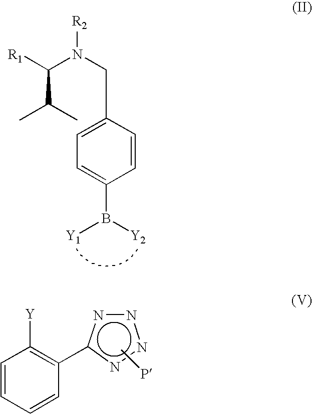

Such reaction is known as Suzuki coupling reaction. It is carried out in an appropriate solvent system and in the presence of a metallic compound. The best conditions to carry out the process vary according to the parameters considered by the person skilled in the art, such as the starting materials, temperature, solvent and similar. Such reaction conditions may be easily determined by the person skilled in the art by routine tests, and with the teaching of the examples included in this document.

Preferably, the metallic compound is selected from palladium, nickel, a metallic salt and a metallic complex. More preferably, the metallic compound is a Pd complex, added or formed in situ, selected from the group consisting of PdX′₂, PdX′₂/PAr₃, PdX′₂/P(C₁C₆)₃, PdX′₂/N(C₁-C₆)₃, PdL₄, and PdX′₂L₂; X′ is a leaving group independently selected from the group consisting of Cl, Br and OCOCH₃; Ar is an aromatic group selected from the group consisting of phenyl, tolyl and furyl; L is a ligand selected from the group consisting of NR′₃, SR′₂, and PR′₃; or alternatively in formula PdX′₂L₂ both L form a diphosphine of formula PR′₂-Z-PR′₂; R′ is independently selected from phenyl, tolyl, furyl, ferrocenyl and (C₁C₆)-alkyl; and Z is selected from ferrocenyl and (C₁-C₄)-alkyl. Still more preferably, the metallic compound is selected from tetrakis(triphenylphosphine)palladium (0), (Pd(PPh₃)₄); dichloro[1,1′-bis(diphenylphosphino)ferrocene]palladium (II), (PdCl₂(dppf); 1,4-bis(diphenylphosphino)butane palladium (II) chloride, (PdCl₂(dppb)); dichlorobis(tricyclohexylphosphine) palladium (II), (PdCl₂(PCy₃)₂); dichloro[1,1′-bis(di-tert-butylphosphino)ferrocene]palladium (II), (PdCl₂(dtbp)); palladium black; palladium (II) chloride; palladium (II) acetate; mixtures of the previously mentioned catalysts with phosphines; and palladium catalysts over polymeric supports. The most preferable metallic compounds are Pd(PPh₃)₄, PdCl₂(dppf), and Pd(OAc)₂/PPh₃.

Preferably, the solvent system is selected from water, an organic solvent selected from aromatic (C₆-C₈) hydrocarbons, an aprotic polar solvent, and aliphatic (C₂-C₈) ethers; and mixtures of water and one or more organic solvents from those mentioned. More preferably the solvent system is selected from tetrahydrofuran and a mixture of dimethylformamide/toluene/water.

Preferably, the base is selected from an alkaline metal carbonate and alkaline metal phosphate. More preferably, the base is potassium carbonate and potassium phosphate.

Preferably, the reaction is carried out at a temperature between ambient temperature and the reflux of the solvent used. More preferably, the reaction is carried out at reflux.

Preferably, the leaving group Y is selected from chlorine, bromine, iodine, methanesulfonyloxy, toluensulfonyloxy, benzenesulfonyloxy or trifluoromethanesulfonyloxy. More preferably, the leaving group is bromine.

When P′ is a protective group P; the process for the preparation of Valsartan includes a deprotection reaction to remove the protective group. A suitable protective group for the tetrazole moiety is selected from those known in the art. Preferably, the protective group is the triphenylmethyl (trityl), but other protective groups can be used for purposes of the present developments. Likewise, the protective group of the tetrazole moiety can be introduced and removed by procedures known in the art (cf. Protective Groups in Organic Synthesis, Wiley-Interscience. (1999)). The specific reaction conditions depend on the protective group used. For instance, when trityl group is used as the protective group of the tetrazole moiety, it can be deprotected either in acidic or basic conditions. Preferably, the deprotection is carried out in acidic, basic or neutral conditions, for example, in methanol or HCl in a suitable solvent such as methanol or a mixture of dioxane/water.

When R₁ is COOR′ with R′=benzyl, p-methoxybenzyl or benzhydryl, the conversion of the ester precursor of valsartan into the free acid form of Valsartan or a salt thereof is carried out by hydrogenolysis in the presence of a suitable hydrogenation catalyst. Examples of suitable hydrogenation catalyst are Pd black, Pd on charcoal, Pd(OH)₂, Pt, PtO₂ and Raney Nickel. When R₁ is COOR′ with R′=t-butyl, the conversion can be carried out by treating the tert-butyl ester with an acid, under mild conditions. When R₁ is COOR′ with R′=methyl or ethyl, the conversion can be achieved by hydrolysis in suitable acid or basic conditions.

An advantage of the present developments lies in the fact that this preparation process allows preparation of Valsartan by a process where the tetrazole moiety, which is easily decomposed in the reaction media, is introduced in the last step. The process of the present developments is particularly advantageous in its practical industrial realization because it avoids the use of azide derivatives which are reactants difficult to handle and also it avoids the use of expensive biphenyl intermediates.

Throughout the description and claims the word “comprise” and variations of the word, such as “comprising”, is not intended to exclude other technical features, additives, components, or steps. The content of the application from which priority is claimed, as well as the contents of the abstracts of the priority application and the present application, are incorporated herein by reference.

Additional objects, advantages and features of the invention will become apparent to those skilled in the art upon examination of the description or may be learned by practice of the invention. The following examples and drawings are provided by way of illustration, and are not intended to be limiting of the present invention.

### EXAMPLES

### Example 1

### Preparation of 4-(5,5-dimethyl-[1,3,2]dioxaborinan-2-yl)-benzaldehyde

To a solution of 4-formylphenylboronic acid (4.11 g) in anhydrous tetrahydrofuran (THF) (40 mL) was added 2,2-dimethyl-1,3-propanediol (3.14 g) and the mixture was stirred at room temperature for 2 hours. The solvent was evaporated to dryness. The residue was dissolved in dichloromethane (120 mL), washed with water (80 mL×3), dried and evaporated under vacuum to obtain 4-(5,5-dimethyl-[1,3,2]dioxaborinan-2-yl)-benzaldehyde (5.66 g, 95% yield).

### Example 2

### Preparation of 4-(5,5-dimethyl-[1,3,2]dioxaborinan-2-yl)-benzaldehyde

To a mixture of 4-formylphenylboronic acid (50 g) in toluene (250 mL) was added 2,2-dimethyl-1,3-propanediol (34.39 g) and the dispersion was heated at reflux for 2 h. The water while formed was azeotropically separated and the residue (330 mL) was used directly in the next step. ¹H-NMR (400 MHz, CDCl₃): δ 1.04 (s, 6 H, 2 CH₃), 3.79 (s, 4 H, 2 CH₂), 7.84 (d, J=6.4 Hz, 2 H, H—Ar), 7.96 (d, J=8 Hz, 2 H, H—Ar), 10.04 (s, 1 H, CHO) ppm.

### Example 3

### Preparation of 4-(4,4,5,5-tetramethyl-[1,3,2]dioxaborolan-2-yl)-benzaldehyde

To a solution of 4-formylphenylboronic acid (1 g) in anhydrous THF (10 mL) was added 2,3-dimethyl-butane-2,3-diol (0.867 mg) and the mixture was stirred at room temperature for 2 hours. The solvent was evaporated to dryness. The residue was dissolved in dichloromethane (40 mL), washed with water (25 mL×3), dried and evaporated under vacuum to obtain 4-(4,4,5,5-tetramethyl-[1,3,2]dioxaborolan-2-yl)-benzaldehyde (1.41 g, 91% yield). ¹H-NMR (400 MHz, CDCl₃): δ 1.34 (s, 12 H, 4 CH₃), 7.86 (d, J=8.4 Hz, 2 H, H—Ar), 7.96 (d, J=8 Hz, 2 H, H—Ar), 10.05 (s, 1 H, CHO) ppm.

### Example 4

### Preparation of methyl N-[4-(5,5-dimethyl-[1,3,2]dioxaborinan-2-yl)Phenyl-4-yl-methyl]-L-valinate

A mixture of L-Valine methyl ester hydrochloride (548 mg), 4-(5,5-dimethyl-[1,3,2]dioxaborinan-2-yl)-benzaldehyde (476 mg) and molecular sieves in anhydrous THF (17 mL) was stirred at room temperature for three days. Then, the mixture was cooled to 0°C. and a solution of NaBH₃CN (155 mg) in dry MeOH (3 mL) was added dropwise. The mixture was stirred at room temperature for 4 hours and the solids were removed by filtration. The filtrate was evaporated under vacuum to yield the desired product (632 mg) that was used in the next reaction without further purification.

### Example 5

### Preparation of methyl N-[4-(5,5-dimethyl-[1.3.2]dioxaborinan-2-yl)phenyl-4-yl-methyl]-L-valinate

A mixture of L-Valine methyl ester hydrochloride (6.77 g), anhydrous MgSO₄ (5.51 g) and Et₃N (5.68 mL) in anhydrous THF (40 mL) was stirred for 15 minutes. To this solution was added while stirring at 0-5° C. 4-(5,5-dimethyl-[1,3,2]dioxaborinan-2-yl)-benzaldehyde (5.87 g) and the mixture was stirred at room temperature for 24 hours. The precipitated salts were removed by filtration and the filtrate evaporated under vacuum. The residue was dissolved in anhydrous methanol (120 mL) and to this solution was added NaBH₄ (1.59g) in small portions, for 1 h. The resulting mixture was stirred at room temperature for 3 hours. Then, was cooled to 0° C. and water (10 mL) was added dropwise. The reaction mixture was concentrated under vacuum, and the residue was partitioned between dichloromethane (100 mL) and water (500 mL). Extraction was carried with dichloromethane (2×50 mL) followed by drying and evaporating to dryness to obtain 8.86 g of the desired product that was used in the next reaction without further purification. ¹H-NMR (400 MHz, CDCl₃): δ 0.92-0.96 (m, 6 H, 2 CH₃ (ⁱPr)), 1.02 (s, 6 H, C(CH₃)₂), 1.77 (bs, 1 H, NH), 1.91 (m, 1 H, CH(CH₃)₂), 3.01 (d, 1 H, J=6.0 Hz, CH—N), 3.59 and 3.85 (2 d,1 H each, J=13 Hz, CH₂-Ph), 3.71 (s, 3 H, CH₃O), 3.76 (s, 4 H, 2CH₂O), 7.32 (d, 2 H, J=8 Hz, H—Ar), 7.75 (d, 2 H, J=8 Hz, H—Ar) ppm.

### Example 6

### Preparation of methyl N-(4-(5,5-dimethyl-[1,3,2]dioxaborinan-2-yl)phenyl-4-yl-methyl)-N-pentanoyl-L-valinate

To a mixture of methyl N-[4-(5,5-dimethyl-[1,3,2]dioxaborinan-2-yl)phenyl-4-yl-methyl]-L-valinate (8.86 g), and Et₃N (4.1 mL) in dichloromethane (170 mL) at 0-5° C. was added dropwise a solution of valeryl chloride (4.9 mL) in dichloromethane (10 mL) and the mixture was stirred at room temperature for 17 hours. Then, triethylamine (4.1 mL) and valeryl chloride (4.9 mL) in dichloromethane (10 mL) were added and the mixture was stirred for 2 h. Water (1 L) was added and extraction was carried out with methylene chloride (2×250 mL), followed by washing with saturated sodium hydrogen carbonate (2×400 mL), drying and evaporating to dryness under reduced pressure. The residue was chromatographed on silica (eluant: cyclohexane/ethyl acetate from 100/0 to 92/8) to obtain methyl N-[4-(5,5-dimethyl-[1,3,2]dioxaborinan-2-yl)phenyl-4-yl-methyl]-N-pentanoyl-L-valinate (9 g, 81% yield, two steps). ¹H-NMR (400 MHz, CDCl₃): Major rotamer: δ 0.8-1.02 (m, 15 H, CH₃ (pentanoyl), 2 CH₃ (ⁱPr), C(CH₃)₂),1.26 (m, 2 H, CH₂Me), 1.60 (m, 2 H, CH₂Et), 2.16-2.36 (m, 3 H, CH₂CO and CH(ⁱPr)), 3.43 (s, 3 H, CH₃O), 3.76 (s, 4 H, CH₂O), 4.62 (s, 2 H, CH₂-Ph), 4.97 (d, 1 H, J=10.4 Hz, CHN), 7.12 (d, 2 H, J=8 Hz, H—Ar), 7.75 (d, 2 H, J=8 Hz, H—Ar) ppm. Minor rotamer: δ 0.8-1.02 (m, 15H, CH₃ (pentanoyl), 2CH₃ (ⁱPr), C(CH₃)₂), 1.40 (m, 2 H, CH₂Me), 1.72 (m, 2 H, CH₂Et), 2.26-2.62 (m, 3 H, CH₂CO and CH(ⁱPr)), 3.34 (s, 3 H, CH₃O), 3.75 (s, 4 H, CH₂O), 4.02 (d, 1 H, J=10.8 Hz, CHN), 4.31 and 4.92 (2 d, 1 H each, J=16 Hz, CH₂-Ph), 7.16 (d, 2 H, J=7.6 Hz, H—Ar), 7.68 (d, 2 H, J=8 Hz, H—Ar) ppm. ¹³C-NMR (100 MHz, CDCl₃): Major rotamer: δ 14.0, 18.9, 20.0 and 22.1 (CH₃), 22.6 (CH₂), 27.7 (CH₂), 27.9 (CH), 31.8 (C), 33.5 (CH₂), 48.5 (CH₂), 51.8 (OCH₃), 61.8 (CH), 72.5 (CH₂), 125.2 and 134.4 (CH—Ar), 139.9 (C-ipso-Ar), 171.3 and 174.9 (CO) ppm. Minor rotamer: δ 13.9, 20.2, 22.1 and 22.4 (CH₃), 22.7 (CH₂), 27 (CH₂), 27.6 (CH), 33.7 (CH₂), 46.0 (CH₂), 51.9 (OCH₃), 66.2 (CH), 72.5 (CH₂), 127 and 133.9 (CH—Ar), 140.8 (C-ipso-Ar), 170.5 and 174.4 (CO) ppm. IR (u): 1740 (CO-ester), 1653 (CO-amide), 1317 (C—O and B—O), 1133 (B—C) cm⁻¹. MS-Cl (NH₃): 418 (M⁺+1, 100), 417 (M⁺, 30).

### Example 7

### Preparation of benzyl N-[4-(5,5-dimethyl-[1,3,2]dioxaborinan-2-yl)phenyl-4-yl-methyl]-L-valinate

A mixture of L-Valine benzyl ester tosylate (0.5 g), anhydrous MgSO₄ (0.18 g) and Et₃N (0.19 mL) in anhydrous THF (6.6 mL) was stirred for 15 minutes. To this solution was added while stirring at 0-5° C. 4-(5,5-dimethyl-[1,3,2]dioxaborinan-2-yl)-benzaldehyde (0.19 g) and the mixture was stirred at room temperature for 24 hours. The precipitated salts were removed by filtration and the filtrate evaporated under vacuum. The residue was dissolved in anhydrous methanol (6.6 mL) and to this solution was added NaBH₄ (52 mg) in small portions, for 1 h. The resulting mixture was stirred at room temperature for 3 hours. Then, was cooled to 0° C. and water (2 mL) was added dropwise. The reaction mixture was concentrated under vacuum, and the residue was partitioned between dichloromethane (15 mL) and water (30 mL). Extraction was carried with dichloromethane (2×5 mL) followed by drying and evaporating to dryness to obtain 426 mg of the desired product that was used in the next reaction without further purification.

### Example 8

### Preparation of (benzyl N-[4-(5,5-dimethyl-[1,3,2]dioxaborinan-2-yl)phenyl-4-yl-methyl]-L-valinate)

A mixture of L-Valine benzyl ester tosylate (132.7 g), 4-(5,5-dimethyl-[1,3,2]dioxaborinan-2-yl)-benzaldehyde (330 mL), Et₃N (48.4 mL) and toluene (413 mL) was heated at reflux temperature for 1 h. The water while formed was azeotropically separated. Then, the mixture was cooled to room temperature and the solution was washed with aqueous NaHCO₃ (317 mL×2) and water (317 mL). The residual water was azeotropically removed. The toluene was partially distilled (134 mL) and MeOH (134 mL) was added. The solution was then cooled to 0-5° C. and NaBH₄ (6.5 g) was slowly added. The reaction mixture was stirred at room temperature overnight. Then the solvent was partially distilled (half volume) and the residue was washed with aqueous NaHCO₃ (270 mL). The separated aqueous phase was extracted with toluene (135 mL×2). The combined organic phases were then washed with water (135 mL). The residual water in the organic layer was azeotropically removed and the residue (aprox. 730 mL) was used directly in the next step. ¹H-NMR (400 MHz, CDCl₃): δ 0.91 and 1.02 (s and m, 12 H, CH₃), 1.77 (bs, 1 H, NH), 1.93 (m, 1 H, CH(CH₃)₂), 1.82 (bs, 1 H, NH), 3.05 (d, 1 H, J=6.4 Hz, CH—N), 3.58 and 3.83 (2 d, J=13.2 Hz, 1 H each, CH₂-Ph), 5.16 (s, 2 H, CH₂-Ph), 7.28 (d, 2 H, J=8 Hz, H—Ar), 7.36 (bs, 5 H, H—Ar), 7.73 (d, 2 H, J=8 Hz, H—Ar) ppm.

### Example 9

### Preparation of benzyl N-[4-(5,5-dimethyl-[1,3,2]dioxaborinan-2-yl)phenyl-4-yl-methyl]-N-pentanoyl-L-valinate

To a mixture of benzyl N-[4-(5,5-dimethyl-[1,3,2]dioxaborinan-2-yl)phenyl-4-yl-methyl]-L-valinate (360 mg), and Et₃N (0.14 mL) in dichloromethane (7 mL) at 0-5° C. was added dropwise a solution of valeryl chloride (0.16 mL) in dichloromethane (0.32 mL) and the mixture was stirred at room temperature for 4 hours. Water (75 mL) and methylene chloride (15 mL) were added and extraction was carried out with methylene chloride (3×5 mL), followed by washing with saturated sodium hydrogen carbonate (2×35 mL), drying and evaporating to dryness under reduced pressure. The residue was chromatographed on silica (eluant: cyclohexane/ethyl acetate from 90/10 to 70/30) to obtain benzyl N-[4-(5,5-dimethyl-[1,3,2]dioxaborina n-2-yl)phenyl-4-yl-methyl]-N-pentanoyl-L-valinate (239 mg, 55% yield, two steps).

### Example 10

### Preparation of benzyl N-[4-(5.5-dimethyl-[1,3,2]dioxaborinan-2-yl)phenyl-4-yl-methyl]-N-pentanoyl-L-valinate)

To the toluene solution of benzyl N-[4-(5,5-dimethyl-[1,3,2]dioxaborinan-2-yl)phenyl-4-yl-methyl]-L-valinate (730 mL) at room temperature was added ethyl-diisopropyl-amine (Hunig base, 66.4 mL) and DMAP (4.03 g). The mixture was then cooled to 0-5° C. and valeryl chloride (42 mL) was added dropwise. The mixture was stirred at room temperature overnight. The reaction mixture was washed with 1 M HCl, followed by washing with water, with saturated sodium hydrogen carbonate, with saturated sodium chloride (488 mL each washing) and with water again (244 mL). The residual water in the organic layer was azeotropically removed and the toluene was evaporated to dryness under reduced pressure to obtain crude benzyl N-[4-(5,5-dimethyl-[1,3,2]dioxaborinan-2-yl)phenyl-4-yl-methyl]-N-pentanoyl-L-valinate (140.1 g, 86% yield, three steps). ¹H-NMR (400 MHz, CDCl₃): Mixture of rotamers: δ 0.8-1.02 (m, 15 H, CH₃ (pentanoyl), 2 CH₃ (ⁱPr), C(CH₃)₂), 1.21-2.6 (m, 7 H, CH₂Me, CH₂Et, CH₂CO and CH(ⁱPr)), 3.76 (s, 4 H, CH₂O), 4.04-4.91 (5 H, N—CH₂-Ph, O—CH₂-Ph and CHN), 7.11-7.71 (9 H, H—Ar) ppm. ¹³C-NMR (100 MHz, CDCl₃): Major rotamer: δ 13.8, 18.9, 19.9 and 21.9 (CH₃), 22.4 (CH₂), 27.4 (CH₂), 28.1 (CH), 31.8 (C), 33.3 (CH₂), 49.9 (CH₂), 62.3 (CH), 66.6 (CH₂), 77.3 (CH₂), 125.2, 126.8, 128.3, 128.4, 128.5, 133.8 and 134.2 (CH—Ar), 135.4 and 139.8 (C-ipso-Ar), 170.4 and 174.7 (CO) ppm. IR (u): 1738 (CO-ester), 1653 (CO-amide), 1317 (C—O and B—O), 1133 (B—C) cm⁻¹. MS-Cl (NH₃): 494 (M⁺+1, 100), 493 (M⁺, 39).

### Example 11

### Preparation of 4-[(1-(S)-methoxycarbonyl-2-methyl-propylamino)-methyl]phenylboronic acid

A mixture of L-Valine methyl ester hydrochloride (885 mg), anhydrous MgSO₄ (960 mg) and Et₃N (0.74 mL) in anhydrous THF (26 mL) was stirred for 15 minutes. To this solution was added while stirring at 0-5° C. 4-formylphenylboronic acid (720 mg) and the mixture was stirred at room temperature overnight. The precipitated salts were removed by filtration and the filtrate evaporated under vacuum. The residue was dissolved in anhydrous methanol (26 mL) and to this solution was added NaBH₄ (284 mg) in small portions, for 1 h. The resulting mixture was stirred at room temperature overnight. Then, was cooled to 0° C. and water (8 mL) was added dropwise. The reaction mixture was concentrated under vacuum, and the residue was partitioned between dichloromethane (70 mL) and water (140 mL). Extraction was carried with dichloromethane (2×20 mL) followed by drying and evaporating to dryness to obtain 703 mg of the desired product that was used in the next reaction without further purification. ¹H-NMR (400 MHz, CDCl₃): δ 0.88-0.89 (2 d, J=5.2 Hz, 3 H each, 2 C(CH₃)₂), 0.94 and 0.96 (2 d, J=6.6 Hz, 3 H each, CH₃ (ⁱPr)), 1.77 (bs, 1 H, NH), 1.92 (m, 1 H, CH(CH₃)₂), 3.03 (d, 1 H, J=6.0 Hz, CH—N), 3.73 (s, 4 H, CH₂O), 3.89 and 3.63 (2 d, J=13.4 Hz, 1 H each, CH₂-Ph,) 7.39 (d, 2 H, J=8 Hz, H—Ar), 8.01 (d, 2 H, J=8 Hz, H—Ar) ppm.

### Example 12

### Preparation of 4-{[(1-(S)-methoxycarbonyl-2-methyl-propyl)-pentanoyl-amino]-methyl}phenylboronic acid

To a mixture of 4-[(1-(S)-Methoxycarbonyl-2-methyl-propylamino)-methyl]phenylboronic acid (224 mg), and Et₃N (0.37 mL) in dichloromethane (4.5 mL) at 0-50° C. was added dropwise a solution of valeryl chloride (0.32 mL) in dichloromethane (0.64 mL) and the mixture was stirred at room temperature for 4 hours. Water (75 mL) was added and extraction was carried out with methylene chloride (1×10 mL and 3×5 mL), followed by washing the organic layer with saturated sodium hydrogen carbonate (5×35 mL), drying and evaporating to dryness under reduced pressure. The residue was chromatographed on silica (eluant: from cyclohexane/ethyl acetate 1:1 to ethyl acetate/MeOH 95:5) to obtain 4-{[-(S)-Methoxycarbonyl-2-methyl-propyl)-pentanoyl-amino]-methyl}phenylboronic acid (191 mg, 65% yield). ¹H-NMR (400 MHz, CDCl₃): δ 0.83-0.99 (m, 9 H, CH₃ (pentanoyl) and 2 CH₃ (ⁱPr)), 1.23-1.79 (m, 4 H, CH₂Me and CH₂Et), 2.19-2.67 (m, 3 H, CH₂CO and CH(ⁱPr)), 3.33-3.47 (4 s, 3 H, CH₃O), 4.07-5.06 (3 H, CH₂-Ph and CHN), 7.16-8.18 (4 H, H—Ar) ppm. ¹³C-NMR (100 MHz, CDCl₃): δ 14, 14.1, 18.9, 20 and 20.1 (CH₃), 22.6, 22.8, 27.6, 27.7, 33.6 (CH₂), 28 and 28.1 (CH), 46.1 and 48.7 (CH₂-Ph), 51.9 and 52.1 (OCH₃), 62 and 66.2 (CH), 125.4, 125.7, 127.2, 135.8 and 136.2 (CH—Ar), 134.1, 134.7, 142.4, 142.6, 143.1 and 143.3 (C-ipso-Ar), 170.5, 171.2, 171.3, 174.6, 175 and 175.2 (CO) ppm. IR (u): 3396 (OH), 1740 (CO-ester), 1635 (CO-amide), 1340 (C—O and B—O) cm⁻¹. MS-ES(+): 350 (M⁺+1).

### Example 13

### Preparation of tert-butyl N-[4-(5,5-dimethyl-[1,3,2]dioxaborinan-2-yl)phenyl-4-yl-methyl]-L-valinate

A mixture of L-Valine tert-butyl ester hydrochloride (1 g), anhydrous MgSO₄ (732 mg) and Et₃N (0.67 mL) in anhydrous THF (24 mL) was stirred for 15 minutes. To this solution was added while stirring at 0-5° C. 4-formylphenylboronic acid (799 mg) and the mixture was stirred at room temperature overnight. The precipitated salts were removed by filtration and the filtrate evaporated under vacuum. The residue was dissolved in anhydrous methanol (24 mL) and to this solution was added NaBH₄ (208 mg) in small portions, for 1 h. The resulting mixture was stirred at room temperature for 3.5 h. Then, was cooled to 0° C. and water (8 mL) was added dropwise. The reaction mixture was concentrated under vacuum, and the residue was partitioned between dichloromethane (35 mL) and water (100 mL). Extraction was carried with dichloromethane (4×15 mL) followed by drying and evaporating to dryness to obtain 1.44 g of the desired product that was used in the next reaction without further purification. ¹H-NMR (400 MHz, CDCl₃): δ 0.94 and 0.95 (2 d, 3 H each, J=6.8 Hz, 2 CH₃ (ⁱPr)), 1.02 (s, 6 H, C(CH₃)₂), 1.48 (s, 9 H, tBu), 1.94 (m, 1 H, CH(CH₃)₂), 2.89 (d, 1 H, J=6.0 Hz, CH—N), 3.65 and 3.89 (2 d, 1 H each, J=13.4 Hz, CH₂-Ph), 3.76 (s, 4 H, 2 CH₂O), 7.35 (d, 2 H, J=8 Hz, H—Ar), 7.75 (d, 2 H, J=8 Hz, H—Ar) ppm.

### Example 14

### Preparation of tert-butyl N-[4-(5,5-dimethyl-[1,3,2]dioxaborinan-2-yl)phenyl-4-yl-methyl]-N-pentanoyl-L-valinate

To a mixture of tert-Butyl N-[4-(5,5-dimethyl-[1,3,2]dioxaborinan-2-yl)phenyl-4-yl-methyl]-L-valinate (1.44 g), and Et₃N (0.59 mL) in dichloromethane (29 mL) at 0-5° C. was added dropwise a solution of valeryl chloride (0.95 mL) in dichloromethane (1.9 mL) and the mixture was stirred at room temperature overnight. Water (146 mL) was added and extraction was carried out with methylene chloride (5×40 mL), followed by washing with saturated sodium hydrogen carbonate, drying and evaporating to dryness under reduced pressure. The residue was chromatographed on silica (eluant: cyclohexane/ethyl acetate from 94/6 to 85/15) to obtain tert-butyl N-[4-(5,5-dimethyl-[1,3,2]dioxaborinan-2-yl)phenyl-4-yl-methyl]-N-pentanoyl-L-valinate (1.56 g, 93% yield, two steps). ¹H-NMR (400 MHz, CDCl₃): mixture of rotamers δ 0.72-1.02 (m, 15 H, CH₃ (pentanoyl), 2 CH₃ (ⁱPr), C(CH₃)₂), 1.22-1.3 (m, 11 H, CH₂Me and CH₃—^{t}Bu), 1.37-1.75 (m, 2 H, CH₂Et), 2.09-2.59 (m, 3 H, CH₂CO and CH(ⁱPr)), 3.75 (2 s, 4 H, CH₂O), 3.9-4.79 (3 H, CH₂-Ph and CHN), 7.18 and 7.21 (2 d, 2 H, J=8 Hz, H—Ar), 7.67 and 7.74 (2 d, 2 H, J=8 Hz, H—Ar) ppm.

### Example 15

### Preparation of methyl N-[4-(4,4,5,5-tetramethyl-[1,3,2]dioxaborolan-2-yl)phenyl-4-yl-methyl]-L-valinate

A mixture of L-Valine methyl ester hydrochloride (670 mg), anhydrous MgSO₄ (612 mg) and Et₃N (0.56 mL) in anhydrous THF (10 mL) was stirred for 15 minutes. To this solution was added while stirring at 0-5° C. 4-(4,4,5,5-tetramethyl-[1,3,2]dioxaborolan-2-yl)-benzaldehyde (710 mg) in anhydrous tetrahydrofuran (10 mL) and the mixture was stirred at room temperature for 24 hours. The precipitated salts were removed by filtration and the filtrate evaporated under vacuum. The residue was dissolved in anhydrous methanol (20 mL) and to this solution was added NaBH₄ (158 mg) in small portions, for 1 h. The resulting mixture was stirred at room temperature for 2 hours. Then, water (4 mL) was added dropwise. The reaction mixture was concentrated under vacuum, and the residue was partitioned between dichloromethane (80 mL) and water (150 mL). Extraction was carried with dichloromethane (2×30 mL) followed by drying and evaporating to dryness to obtain 981 mg of the desired product that was used in the next reaction without further purification. ¹H-NMR (400 MHz, CDCl₃): δ 0.92 and 0.94 (2 d, 3 H each, J=6.8 Hz, 2 CH₃ (ⁱPr)), 1.34 (S, 12 H, C(CH₃J₂), 1-91 (m, 1 H, CH(CH₃)₂), 3.49 (d, 1 H₁ J=6.4 Hz, CH—N), 3.59 and 3.86 (2 d, 1 H each, J=13.4 Hz, CH₂-Ph), 3.71 (s, 3 H, CH₃O), 7.34 (d, 2 H, J=8 Hz, H—Ar), 7.76 (d, 2 H, J=8 Hz, H—Ar) ppm.

### Example 16

### Preparation of methyl N-[4-(4,4,5,5-tetramethyl-[1,3,2]dioxaborolan-2-yl)phenyl-4-yl-methyl]-N-Pentanoyl-L-valinate

To a mixture of methyl N-[4-(4,4,5,5-tetramethyl-[1,3,2]dioxaborolan-2-yl)phenyl-4-yl-methyl]-L-valinate (980 mg), and Et₃N (0.44 mL) in dichloromethane (20 mL) at 0-5° C. was added dropwise a solution of valeryl chloride (0.52 mL) in dichloromethane (1 mL) and the mixture was stirred at room temperature for 4 hours. Then, triethylamine (0.4 mL) and valeryl chloride (0.35 mL) were added and the mixture was stirred for 3 days. Water (100 mL) was added and extraction was carried out with methylene chloride (1×25 mL and 2×10 mL), followed by washing with saturated sodium hydrogen carbonate (2×50 mL), drying and evaporating to dryness under reduced pressure. The residue was chromatographed on silica (eluant: cyclohexane/ethyl acetate from 95/5 to 85/15) to obtain methyl N-[4-(4,4,5,5-tetramethyl-[1,3,2]dioxaborolan-2-yl)phenyl-4-yl-methyl]-N-pentanoyl-L-valinate (695 mg, 57% yield, two steps). ¹H-NMR (400 MHz, CDCl₃): Mixture of rotamers: δ 0.82-0.97 (m, 9 H, CH₃ (pentanoyl) and 2 CH₃ (ⁱPr)), 1.24-1.34 (m, 14 H, 2 C(CH₃)₂ and CH₂Me), 1.57-1.75 (m, 2 H, CH₂Et), 2.14-2.6 (m, 3 H, CH₂CO and CH(ⁱPr)), 3.37 and 3.43 (2 s, 3 H each, CH₃O), 4.03 (d, 1 H, J=10.4 Hz, CHN minor rotamer), 4.33 and 4.89 (2 d, 1 H each, J=15.4 Hz, CH₂-Ph minor rotamer), 4.63 (s, 2 H, CH₂-Ph major rotamer), 4.98 (d, 1 H, J=10.4 Hz, CHN major rotamer), 7.14 (d, 2 H, J=7.8 Hz, H—Ar major rotamer), 7.17 (d, 2 H, J=7.6 Hz, H—Ar minor rotamer), 7.76 (d, 2 H, J=7.8 Hz, H—Ar major rotamer) 7.79 (d, 2 H, J=7.6 Hz, H—Ar minor rotamer) ppm. ¹³C-NMR (100 MHz, CDCl₃): Major rotamer: δ 13.8, 18.6, 19.8 and 24.8 (CH₃), 22.4 (CH₂), 27.4 (CH₂), 27.8 (CH), 33.3 (CH₂), 48.2 (CH₂), 51.6 (OCH₃), 61.6 (CH), 83.8 (C), 125 and 135.1 (CH—Ar), 140.5 (C-ipso-Ar), 171 and 174.7 (CO) ppm. IR (u): 1741 (CO-ester), 1654 (CO-amide), 1361 (C—O and B—O), 1145 (B—C) cm⁻¹.

### Example 17

### Preparation of methyl N-pentanoyl-N-[{2′-[1-(triphenylmethyl)-1H-tetrazol-5-yl]-1,1′-biphenyl-4-yl}methyl]-L-valinate

A Schlenk tube was charged with methyl N-[4-(5,5-dimethyl-[1,3,2]dioxaborinan-2-yl)phenyl-4-yl-methyl]-N-pentanoyl-L-valinate (150 mg), 5-(2-bromophenyl)-1-triphenylmethyl-1H-tetrazole (98 mg), anhydrous K₃PO₄ (153 mg) and anhydrous tetrahydrofuran (2.5 mL). The suspension was degassed by vacuum/nitrogen purges (3×). Tetrakis(triphenylphosphine)palladium (0) (Pd(PPh₃)₄) (13 mg) was added, the mixture was degassed by vacuum/nitrogen purges (3×) and refluxed for 48 h. After cooling to room temperature, the reaction mixture was passed through a pad of celite®, the cake was washed with methylene chloride and ethyl acetate. The filtrate and washings were combined and evaporated to dryness. The residue was purified by column chromatography (eluant: cyclohexane/ethyl acetate, 8:2) to give methyl N-pentanoyl-N-[{2′-[1-(triphenylmethyl)-1H-tetrazol-5-yl]-1,1′-biphenyl-4-yl}methyl]-L-valinate (94 mg, 54% yield).

### Example 18

### Preparation of methyl N-pentanoyl-N-[{2′-[1-(triphenylmethyl)-1H-tetrazol-5-yl]-1,1′-biphenyl-4-yl}methyl]-L-valinate

A mixture of methyl N-[4-(5,5-dimethyl-[1,3,2]dioxaborinan-2-yl)phenyl-4-yl-methyl]-N-pentanoyl-L-valinate (2 g), 5-(2-Bromophenyl)-1-triphenylmethyl-1H-tetrazole (2.24 g) and anhydrous K₃PO₄ (3.11 g) in anhydrous tetrahydrofuran (33 mL) was degassed by vacuum/nitrogen purges (3×). Then, dichloro[1,1′-bis(diphenylphosphino)ferrocene]palladium (II) (117 mg) was added, the mixture was degassed by vacuum/nitrogen purges (3×) and heated at reflux for 4 days. The reaction mixture was cooled, passed through a pad of celite® and the filtrate evaporated under vacuum. The residue was chromatographed on silica (eluant: cyclohexane/ethyl acetate 9:1) to give methyl N-pentanoyl-N-[{2′-[1-(triphenylmethyl)-1H-tetrazol-5-yl]-1,1′-biphenyl-4-yl}methyl]-L-valinate (2.53 g, 76% yield).

### Example 19

### Preparation of methyl N-pentanoyl-N-[{2′-[1-(triphenylmethyl)-1H-tetrazol-5-yl]-1,1′-biphenyl-4-yl}methyl]-L-valinate

A mixture of methyl N-[4-(5,5-dimethyl-[1,3,2]dioxaborinan-2-yl)phenyl-4-yl-methyl]-N-pentanoyl-L-valinate (200 mg), 5-(2-Bromophenyl)-1-triphenylmethyl-1H-tetrazole (224 mg) and anhydrous K₂CO₃ (199 mg) in anhydrous tetrahydrofuran (3.3 mL) was degassed by vacuum/nitrogen purges (3×). Then, dichloro[1,1′-bis(diphenylphosphino)ferrocene]palladium (II) was added, the mixture was degassed by vacuum/nitrogen purges (3×) and heated at reflux for 48 hours. The reaction mixture was cooled, passed through a pad of celite® and the filtrate evaporated under vacuum. The residue was chromatographed on silica (eluant: cyclohexane/ethyl acetate: 9/1) to give methyl N-pentanoyl-N-[{2′-[1-(triphenylmethyl)-1H-tetrazol-5-yl]-1,1′-biphenyl-4-yl}methyl]-L-valinate (185 mg, 56% yield).

### Example 20

### Preparation of methyl N-pentanoyl-N-[{2′-[1-(triphenylmethyl)-1H-tetrazol-5-yl]-1,1′-biphenyl-4-yl}methyl]-L-valinate

A Schlenk tube was charged with methyl N-[4-(5,5-dimethyl-[1,3,2]dioxaborinan-2-yl)phenyl-4-yl-methyl]-N-pentanoyl-L-valinate (150 mg), 5-(2-bromophenyl)-1-triphenylmethyl-1H-tetrazole (140 mg), anhydrous K₂CO₃ (210 mg), toluene (2 mL), dimethylformamide (0.2 mL) and H₂O (0.6 mL). The suspension was purged and degassed. Dichloro[1,1′-bis(diphenylphosphino)ferrocene]-palladium (II) (PdCl₂(dppf)) was added, the mixture was purged and degassed. The reaction was heated at 85° C. for 48 h. After cooling to room temperature, ethyl acetate (3 mL) and H₂O (0.4 mL) were added, the mixture was stirred for 10 min. The organic layer was washed with H₂O (3 mL), dried over anhydrous Na₂SO₄, filtered and evaporated to dryness. The residue was purified by column chromatography (eluant: cyclohexane/ethyl acetate, 9:1) to give methyl N-pentanoyl-N-[{2′-[1-(triphenylmethyl)-1H-tetrazol-5-yl]-1,1′-biphenyl-4-yl}methyl]-L-valinate (21 mg).

### Example 21

### Preparation of methyl N-pentanoyl-N-[{2′-[1-(triphenylmethyl)-1H-tetrazol-5-yl]-1,1′-biphenyl-4-yl}methyl]-L-valinate

A Schlenk tube was charged with crude 4-{[(1-(S)-methoxycarbonyl-2-methyl-propyl)-pentanoyl-amino]-methyl}phenylboronic acid (200 mg), 5-(2-Bromophenyl)-1-triphenylmethyl-1H-tetrazole (224 mg), anhydrous K₂CO₃ (334 mg), toluene (3 mL), dimethylformamide (0.3 mL) and H₂O (0.94 mL). The suspension was purged and degassed. Dichloro[1,1′-bis(diphenylphosphino)ferrocene]palladium (II) (PdCl₂(dppf)) was added, the mixture was purged and degassed. The reaction was heated at 85 ° C. for 48 h. After cooling to room temperature, ethyl acetate (4 mL) and H₂O (0.6 mL) were added, the mixture was stirred for 10 min. The aqueous layer was extracted with ethyl acetate (2 mL×2). The organic layer was washed with H₂O (8 mL), dried over anhydrous Na₂SO₄, filtered and evaporated to dryness. The residue was purified by column chromatography (eluant: cyclohexane/ethyl acetate, 9:1) to give methyl N-pentanoyl-N-[{2′-[1-(triphenylmethyl)-1H-tetrazol-5-yl]-1,1′-biphenyl-4-yl}methyl]-L-valinate (53 mg).

### Example 22

### Preparation of methyl N-pentanoyl-N-[{2′-[1-(triphenylmethyl)-1H-tetrazol-5-yl]-1,1′-biphenyl-4-yl}methyl]-L-valinate

A solution of PPh₃ (5 mg) in anhydrous tetrahydrofuran (5 mL) was degassed by vacuum/nitrogen purges (3×). Then, Pd(OAc)₂ (4 mg) was added and the mixture was stirred for 30 min after degassing by vacuum/nitrogen purges (3×). Methyl N-[4-(5,5-dimethyl-[1,3,2]dioxaborinan-2-yl)phenyl-4-yl-methyl]-N-pentanoyl-L-valinate (200 mg), 5-(2-bromophenyl)-1-triphenylmethyl-1H-tetrazole (224 mg) and anhydrous K₃PO₄ (312 mg) were added. The mixture was degassed by vacuum/nitrogen purges (3×) and heated at reflux for 48 hours. The reaction mixture was cooled, passed through a pad of celite® and the filtrate evaporated under vacuum. The residue was chromatographed on silica (eluant: cyclohexane/ethyl acetate: 9/1) to give methyl N-pentanoyl-N-[{2′-[1-(triphenylmethyl)-1H-tetrazol-5-yl]-1,1′-biphenyl-4-yl}methyl]-L-valinate (119 mg, 36% yield).

### Example 23

### Preparation of methyl N-Pentanoyl-N-[{2′-[1-(triphenylmethyl)-1H-tetrazol-5-yl]-1,1′-biphenyl-4-yl}methyl]-L-valinate

A solution of PPh₃ (15 mg) in anhydrous THF (3.3 mL) was degassed by vacuum/nitrogen purges (3×). Then, anchored homogeneous palladium catalyst (57 mg) was added and the mixture was stirred for 30 min after degassing by vacuum/nitrogen purges (3×). Methyl N-[4-(5,5-dimethyl-[1,3,2]dioxaborinan-2-yl)phenyl-4-yl-methyl]-N-pentanoyl-L-valinate (200 mg), 5-(2-Bromophenyl)-1-triphenylmethyl-1H-tetrazole (374 mg) and anhydrous K₃PO₄ (311 mg) were added. The mixture was degassed by vacuum/nitrogen purges (3×) and heated at reflux for 48 hours. The reaction mixture was cooled, passed through a pad of celite® and the filtrate evaporated under vacuum. The residue was chromatographed on silica (eluant: cyclohexane/ethyl acetate: 9:1) to give methyl N-pentanoyl-N-[{2′-[1-(triphenylmethyl)-1H-tetrazol-5-yl]-1,1′-biphenyl-4-yl}methyl]-L-valinate (224 mg, 67% yield).

### Example 24

### Preparation of methyl N-pentanoyl-N-[{2′-[1-(triphenylmethyl)-1H-tetrazol-5-yl]-1,1′-biphenyl-4-yl}methyl]-L-valinate

A solution of PPh₃ (15 mg) in anhydrous THF (3.3 mL) was degassed by vacuum/nitrogen purges (3×). Then, anchored homogeneous palladium catalyst (37 mg) was added and the mixture was stirred for 30 min after degassing by vacuum/nitrogen purges (3×). Methyl N-[4-(5,5-dimethyl-[1,3,2]dioxaborinan-2-yl)phenyl-4-yl-methyl]-N-pentanoyl-L-valinate (200 mg), 5-(2-Bromophenyl)-1-triphenylmethyl-1H-tetrazole (374 mg) and anhydrous K₃PO₄ (311 mg) were added. The mixture was degassed by vacuum/nitrogen purges (3×) and heated at reflux for 48 hours. The reaction mixture was cooled, passed through a pad of celite® and the filtrate evaporated under vacuum. The residue was chromatographed on silica (eluant: cyclohexane/ethyl acetate: 9:1) to give methyl N-pentanoyl-N-[{2′-[1-(triphenylmethyl)-1H-tetrazol-5-yl]-1,1′-biphenyl-4-yl}methyl]-L-valinate (242 mg, 73% yield).

### Example 25

### Preparation of methyl N-pentanoyl-N-[{2′-[1-(triphenylmethyl)-1H-tetrazol-5-yl]-1,1′-biphenyl-4-yl}methyl]-L-valinate

A mixture of methyl N-[4-(4,4,5,5-tetramethyl-[1,3,2]dioxaborolan-2-yl)phenyl-4-yl-methyl]-N-pentanoyl-L-valinate (200 mg), 5-(2-Bromophenyl)-1-triphenylmethyl-1H-tetrazole (215 mg) and anhydrous K₃PO₄ (295 mg) in anhydrous tetrahydrofuran (3.3 mL) was degassed by vacuum/nitrogen purges (3×). Then, dichloro[1,1′-bis(diphenylphosphino)ferrocene]palladium (II) (11 mg) was added, the mixture was degassed by vacuum/nitrogen purges (3×) and heated at reflux for 48 hours. The reaction mixture was cooled, passed through a pad of celite® and the filtrate evaporated under vacuum. The residue was chromatographed on silica (eluant: cyclohexane/ethyl acetate: 9/1) to give methyl N-pentanoyl-N-[{2′-[1-(triphenylmethyl)-1H-tetrazol-5-yl]-1,1′-biphenyl-4-yl}methyl]-L-valinate_(52 mg). ¹H-NMR (400 MHz, CDCl₃): Major rotamer: δ 0.78-0.98 (m, 9 H, 2 CH₃ (ⁱPr), CH₃ (pentanoyl)), 1.20 (m, 2 H, CH₂Me), 1.56 (m, 2 H, CH₂Et), 2.09-2.37 (m, 3 H, CH₂CO and CH(ⁱPr)), 3.36 (s, 3H, CH₃O), 4.52 (s, 2 H, CH₂-Ph), 4.82 (d, 1 H, J=10 Hz, CHN), 6.95-7.86 (m, 23 H, H—Ar) ppm. Minor rotamer: δ 0.78-0.98 (m, 9 H, 2 CH₃ (ⁱPr), CH3 (pentanoyl)), 1.39 (m, 2 H, CH₂Me), 1.73 (m, 2 H, CH₂Et), 2.27-2.59 (m, 3 H, CH₂CO and CH(ⁱPr)), 3.28 (s, 3 H, CH₃O), 3.99 (d, 1 H, J=10.8 Hz, CHN), 4.19 and 4.86 (2 d, 1 H each, J=15.2 Hz, CH₂-Ph), 6.95-7.86 (m, 23 H, H—Ar) ppm. ¹³C-NMR (100 MHz, CDCl₃): Major rotamer: δ 13.8, 18.8 and 19.9 (CH₃), 22.4 (CH₂), 27.4 (CH₂), 27.5 (CH), 33.3 (CH₂), 48.5 (CH₂), 51.5 (OCH₃), 61.9 (CH), 125.6-141.6 (CH— and C-ipso-Ar), 171.0 and 174.5 (CO) ppm. Minor rotamer: δ 13.6, 19.9 and 22.1 (CH₃), 22.5 (CH₂), 26.8 (CH₂), 27.5 (CH), 29.7 (CH₂), 45.4 (CH₂), 51.8 (OCH₃), 65.8 (CH), 125.6-141.6 (CH— and C-ipso-Ar), 170.3 and 174.5 (CO) ppm.

### Example 26

### Preparation of benzyl N-pentanoyl-N-[{2′-[1-(triphenylmethyl)-1H-tetrazol-5-yl]-1,1′-biphenyl-4-yl}methyl]-L-valinate

A mixture of benzyl N-[4-(5,5-dimethyl-[1,3,2]dioxaborinan-2-yl)phenyl-4-yl-methyl]-N-pentanoyl-L-valinate (360 mg), 5-(2-Bromophenyl)-1-triphenylmethyl-1H-tetrazole (341 mg) and anhydrous K₃PO₄ (464 mg) in anhydrous tetrahydrofuran (6 mL) was degassed by vacuum/nitrogen purges (3×). Then, dichloro[1,1′-bis(diphenylphosphino)ferrocene]palladium (II) (18 mg) was added, the mixture was degassed by vacuum/nitrogen purges (3×) and heated at reflux for 48 hours. The reaction mixture was cooled, passed through a pad of celite® and the filtrate evaporated under vacuum. The residue was chromatographed on silica (eluant: cyclohexane/ethyl acetate: 95/5) to give benzyl N-pentanoyl-N-[{2′-[1-(triphenylmethyl)-1H-tetrazol-5-yl]-1,1′-biphenyl-4-yl}methyl]-L-valinate (279 mg, 41% yield).

### Example 27

### Preparation of benzyl N-pentanoyl-N-[{2′-[1-(triphenylmethyl)-1H-tetrazol-5-yl]-1,1′-biphenyl-4-yl}methyl]-L-valinate

A solution of Pd(OAc)₂ (9 mg) and PPh₃ (40 mg) in toluene (5 mL) and tetrahydrofuran (3 mL) was degassed by argon purge (5 min.) and stirred at room temperature for 30 min. under argon atmosphere. Then, 5-(2-Bromophenyl)-1-triphenylmethyl-1H-tetrazole (1.8 g), anhydrous K₃PO₄ (1.25 g) and a solution of benzyl N-[4-(5,5-dimethyl-[1,3,2]dioxaborinan-2-yl)phenyl-4-yl-methyl]-N-pentanoyl-L-valinate (2 g) in toluene (7 mL) were added. The mixture was degassed by argon purge (5 min.) and heated at 85° C. overnight. The reaction mixture was cooled, and water (5 mL) was added. The aqueous layer was extracted with toluene (3.5 mL×2). The water of the combined organic layers was azeotropically removed and the solvent was evaporated to dryness under reduced pressure to obtain crude benzyl N-pentanoyl-N-[{2′-[1-(triphenylmethyl)-1H-tetrazol-5-yl]-1,1′-biphenyl-4-yl}methyl]-L-valinate (3.07 g, quantitative). ¹H-NMR (400 MHz, CDCl₃): Major rotamer: δ 0.76-0.97 (m, 9 H, 2 CH₃ (ⁱPr), CH₃ (pentanoyl)), 1.13-1.72 (m, 4 H, CH₂Me and CH₂Et), 2.09-2.59 (m, 3 H, CH₂CO and CH(ⁱPr)), 4.43 and 4.57 (2 d, 1 H each, J=17.4 Hz, CH2—N), 4.79 (d, 1 H, J=10.8 Hz, CHN), 4.81 and 4.87 (2 d, 1 H each, J=12.2 Hz, CH₂-Ph), 6.94-7.87 (m, 23 H, H—Ar) ppm. Minor rotamer: δ 0.76-0.97 (m, 9 H, 2 CH₃ (ⁱPr), CH₃ (pentanoyl)), 1.13-1.72 (m, 4 H, CH₂Me and CH₂Et), 2.09-2.59 (m, 3 H, CH₂CO and CH(ⁱPr)), 4.03 (d, 1 H, J=10.4 Hz, CHN), 4.27 and 4.64 (2 d, 1 H each, J=15.2 Hz, CH₂-Ph), 4.64 and 4.81 (2 d, 1 H each, J=9.6 Hz, CH₂-Ph), 6.94-7.87 (m, 23 H, H—Ar) ppm. ¹³C-NMR (100 MHz, CDCl₃): Major rotamer: δ 13.8, 19 and 20.2 (CH₃), 22.3 (CH₂), 27.4 (CH₂), 28.1 (CH), 33.3 (CH₂), 49.1 (CH₂), 62.8 (CH), 66.5 (CH₂), 125.8-141.6 (CH— and C-ipso-Ar), 170.3 and 174.3 (CO) ppm. Minor rotamer: δ 13.8, 18.8 and 19.9 (CH₃), 22.5 (CH₂), 26.9 (CH), 27.4 (CH₂), 33.5 (CH₂), 45.5 (CH₂), 66.1 (CH), 66.8 (CH₂), 125.8-141.6 (CH— and C-ipso-Ar) ppm. IR (u): 1734 (CO-ester), 1654 (CO-amide) cm⁻¹.

### Example 28

### Preparation of (N-pentanoyl-N-[{2′-[1-(triphenylmethyl)-1H-tetrazol-5-yl]-1,1′-biphenyl-4-yl}methyl]-L-valine)

A mixture of benzyl N-pentanoyl-N-[{2′-[1-(triphenylmethyl)-1H-tetrazol-5-yl]-1,1′-biphenyl-4-yl}methyl]-L-valinate (0.3 g) in AcOEt (3 mL) containing 5% palladium on activated Charcoal (Pd/C, 34 mg ) was hydrogenated at room temperature for 3h. The resulting crude product was filtered through a Celite® pad and the solvent was evaporated to dryness to furnish the desired product (0.23 g, 86%). ¹H-NMR (400 MHz, CDCl₃): δ 0.85 and 0.98 (2 d, 3 H each, J=6.8 Hz, 2 CH₃ (ⁱPr)), 0.88 (t, 3 H, J=7.2 Hz, CH₃ (pentanoyl)), 1.29 (m, 2 H, CH₂Me), 1.61 (m, 2 H, CH₂Et), 2.36 (m, 2 H, CH₂CO), 2.74 (m, 1 H, CH(ⁱPr)), 3.51 (d, 1 H, J=10.8 Hz, CHN), 4.28 and 4.60 (2 d, 1 H each, J=16.2 Hz, CH₂-N), 6.94-7.92 (m, 23 H, H—Ar) ppm. ¹³C-NMR (100 MHz, CDCl₃): δ 13.8, 19.5 and 19.7 (CH₃), 22.3 (CH₂), 27.0 (CH₂), 27.1 (CH), 34.1 (CH₂), 54.8 (CH₂), 72.6 (CH), 82.8 (C), 126.1-141.3 (CH— and C-ipso-Ar), 164.0 (CN), 171.4 and 177.3 (CO) ppm. IR (u): 2960 (broad band, OH), 1741 (CO-acid), 1610 (CO-amide) cm⁻¹. Mp 170° C.

### Example 29

### Preparation of tert-butyl N-pentanoyl-N-[{2′-[1-(triphenylmethyl)-1H-tetrazol-5-yl]-1,1′-biphenyl-4-yl}methyl]-L-valinate

A mixture of tert-butyl N-[4-(5,5-dimethyl-[1,3,2]dioxaborinan-2-yl)phenyl-4-yl-methyl]-N-pentanoyl-L-valinate (1.56 g), 5-(2-Bromophenyl)-1-triphenylmethyl-1H-tetrazole (1.59 g) and anhydrous K₃PO₄ (2.16 g) in anhydrous tetrahydrofuran (26 mL) was degassed by vacuum/nitrogen purges (3×). Then, dichloro[1,1′-bis(diphenylphosphino)ferrocene]palladium (II) (83 mg) was added, the mixture was degassed by vacuum/nitrogen purges (3×) and heated at reflux for 2 days. The reaction mixture was cooled, passed through a pad of celite® and the filtrate evaporated under vacuum. The residue was chromatographed on silica (eluant: cyclohexane/ethyl acetate 95:5) to give tert-butyl N-pentanoyl-N-[{2′-[1-(triphenylmethyl)-1H-tetrazol-5-yl]-1,1′-biphenyl-4-yl}methyl]-L-valinate_(1.65 g, 66% yield). ¹H-NMR (400 MHz, CDCl₃): mixture of rotamers δ 0.69-0.98 (m, 9 H, 2 CH₃ (ⁱPr), CH₃ (pentanoyl)), 1.14-1.45 (m, 11 H, CH₂Me and CH₃—^{t}Bu), 1.47-1.76 (m, 2 H, CH₂Et), 2.1-2.61 (m, 3 H, CH₂CO and CH(ⁱPr)), 3.86-4.67 (3 H, CH₂-Ph and CHN), 6.93-7.87 (m, 23 H, H—Ar) ppm. IR (u): 1728 (CO-ester), 1654 (CO-amide) cm⁻¹.

### Example 30

### Preparation of methyl N-pentanoyl-N-[{2′-(1H-tetrazol-5-yl)-1,1′-biphenyl-4-yl]methyl}-L-valinate

A solution of methyl N-pentanoyl-N-[{2′-[1-(triphenylmethyl)-1H-tetrazol-5-yl]-1,1′-biphenyl-4-yl}methyl]-L-valinate (164 mg) in MeOH (6 mL) was heated at reflux. Then, 1 M HCl (6 mL) was added dropwise and the mixture was stirred for 3.5 h. The reaction mixture was evaporated under vacuum and the residue was partitioned between aqueous NaHCO₃ (5 mL) and ethyl acetate (10 mL). Extraction was carried with ethyl acetate (3×10 mL) followed by drying and evaporating to dryness. The residue was chromatographed on silica (eluant: hexane/ethyl acetate from 100:0 to 0:100) to obtain methyl N-pentanoyl-N-{[2′-(1H-tetrazol-5-yl)-1,1′-biphenyl-4-yl]methyl}-L-valinate (101 mg, 95%). ¹H-NMR (400 MHz, CDCl₃): Major rotamer: δ 0.73-0.91 (m, 9 H, 2 CH₃ (ⁱPr), CH₃ (pentanoyl)), 1.26 (m, 2 H, CH₂Me), 1.51 (m, 2 H, CH₂Et), 2.07-2.52 (m, 3 H, CH₂CO and CH(ⁱPr)), 3.35 (s, 3H, CH₃O), 4.50 and 4.56 (2 d, 1 H each, J=17.8 Hz, CH₂-Ph), 4.76 (d, 1 H, J=10.4 Hz, CHN), 6.93-7.84 (m, 8 H, H—Ar) ppm. Minor rotamer: δ 0.73-0.91 (m, 9 H, 2 CH₃ (ⁱPr), CH₃ (pentanoyl)), 1.18 (m, 2 H, CH₂Me), 1.51 (m, 2 H, CH₂Et), 2.07-2.52 (m, 3 H, CH₂CO and CH(ⁱPr)), 3.29 (s, 3 H, CH₃O), 3.97 (d, 1 H, J=10.8 Hz, CHN), 4.16 and 4.82 (2 d, 1 H each, J=15.4 Hz, CH₂-Ph), 6.93-7.84 (m, 8 H, H—Ar) ppm.

### Example 31

### Preparation of benzyl N-pentanoyl-N-{[2′-(1H-tetrazol-5-yl)-1,1′-biphenyl-4-yl]methyl}-L-valinate

A solution of benzyl N-pentanoyl-N-[{2′-[1-(triphenylmethyl)-1H-tetrazol-5-yl]-1,1′-biphenyl-4-yl}methyl]-L-valinate_(330 mg) in MeOH (6 mL) was heated at reflux for 2 h and at room temperature overnight. The reaction mixture was evaporated under vacuum and the residue was chromatographed on silica (eluant: cyclohexane/ethyl acetate from 7:3 to 3:7) to obtain benzyl N-pentanoyl-N-{[2′-(1H-tetrazol-5-yl)-1,1′-biphenyl-4-yl]methyl}-L-valinate_(207 mg, 92%). ¹H-NMR (400 MHz, CDCl₃): Major rotamer: δ 0.84-0.99 (m, 9 H, 2 CH₃ (ⁱPr), CH₃ (pentanoyl)), 1.29 (m, 2 H, CH₂Me), 1.62 (m, 2 H, CH₂Et), 2.19-2.61 (m, 3 H, CH₂CO and CH(ⁱPr)), 4.1 (d, 1 H, J=10.8 Hz, CHN), 4.62 and 4.72 (2 d,1 H each, J=17.6 Hz, CH₂-Ph), 4.86 and 4.95 (2 d, 1 H each, J=12.2 Hz, OCH₂-Ph), 7.06-8.21 (m, 13 H, H—Ar) ppm. Minor rotamer: δ 0.84-0.99 (m, 9 H, 2 CH₃ (ⁱPr), CH₃ (pentanoyl)), 1.29 (m, 2 H, CH₂Me), 1.62 (m, 2 H, CH₂Et), 2.19-2.61 (m, 3 H, CH₂CO and CH(ⁱPr)), 4.34 and 4.98 (2 d, 1 H each, J=15.6 Hz, CH₂-Ph), 4.76 (d, 1 H, J=10 Hz, CHN), 4.88 and 4.95 (2 d, 1 H each, J=12.2 Hz, OCH₂-Ph), 7.06-8.21 (m, 13 H, H—Ar) ppm.

### Example 32

### Preparation of N-pentanoyl-N-{[2′-(1H-tetrazol-5-yl)-1,1′-biphenyl-4-yl]methyl}-L-valine (VALSARTAN)

A mixture of methyl N-pentanoyl-N-{[2′-(1H-tetrazol-5-yl)-1,1′-biphenyl-4-yl]methyl}-L-valinate (50 mg) in 10% NaOH (1 mL) was stirred at room temperature overnight. The mixture was acidified with concentrated HCI and extraction was carried with ethyl acetate (4×10 mL). The organic layer was washed with saturated NaCl solution, followed by drying and evaporating to dryness (42 mg, 88%).

### Example 33

### Preparation of N-pentanoyl-N-{[2′-(1H-tetrazol-5-yl)-1,1′-biphenyl-4-yl]methyl}-L-valine (VALSARTAN)

A mixture of benzyl N-pentanoyl-N-{[2′-(1H-tetrazol-5-yl)-1,1′-biphenyl-4-yl]Methyl}-L-valinate_(200 mg) in MeOH (2 mL) containing 10% palladium on activated Charcoal (Pd/C, 40 mg) was hydrogenated at room temperature for 5 h. The resulting crude product was filtered through a Celite® pad and the solvent was evaporated. The crude was partitioned between diethyl ether and 2M NaOH. The aqueous layer was then acidified with concentrated HCl. Extraction was carried with ethyl acetate followed by drying and evaporating to dryness to furnish the desired product (91 mg, 55%). ¹H-NMR (400 MHz, CDCl₃): Major rotamer: δ 0.94-1.11 (m, 9 H, 2 CH₃ (ⁱPr), CH₃ (pentanoyl)), 1.42 (m, 2 H, CH₂Me), 1.73 (m, 2 H, CH₂Et), 2.43 and 2.64 (2 m, 3 H, CH₂CO and CH(ⁱPr)), 4.17 (d, 1 H, J=10.4 Hz, CHN), 4.36 and 4.98 (2 d, 1 H each, J=15.6 Hz, CH₂-Ph), 7.07-7.99 (m, 8 H, H—Ar) ppm. Minor rotamer: δ 0.94-1.11 (m, 9 H, 2 CH₃ (ⁱPr), CH₃ (pentanoyl)), 1.42 (m, 2 H, CH₂Me), 1.73 (m, 2 H, CH₂Et), 2.43 and 2.64 (2 m, 3 H, CH₂CO and CH(ⁱPr)), 3.68 (bs, 1 H, CHN), 4.30 and 5.18 (2 d, 1 H each, J=15.6 Hz, CH₂-Ph), 7.07-7.99 (m, 8 H, H—Ar) ppm. IR (u): 3000 (broad band, OH), 1782 (CO-acid), 1722 (CO-amide) cm⁻¹. MS-Cl (NH₃): 436 (M⁺+1, 22).

1. A compound of formula (II), 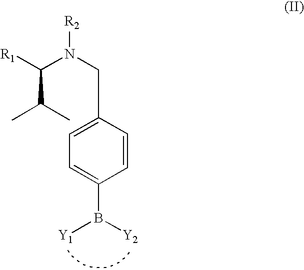
wherein:
Y₁ and Y₂ are each independently selected from the group consisting of hydroxy, (C₁-C₄)-alcoxy and phenoxy, the latter optionally substituted by a (C₁-C₄)-alcoxy, (C₁-C₄)-alkyl or a halogen group; or alternatively Y₁ and Y₂ can be taken together with a boron atom to form a cyclic structure selected from the following ones; 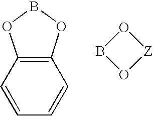
wherein Z is selected from the group consisting of (CH₂)ₙ, (CH₂)ᵣCRᵤRᵥ(CH₂)ₛ and CRᵤRᵥ(CH₂)ₜCRᵤRᵥ; n is an integer from 2 to 4; r and s are integers from 0 to 4 with the condition that r and s are not both 0; t is an integer from 0 to 1, and Rᵤ and Rᵥ are each independently selected from the group consisting of H, (C₁-C₄)-alkyl, phenyl and mono- or di-substituted phenyl, the substituents being halogen, (C₁-C₄)-alkyl and (C₁-C₄)-alkoxy;
R₁ represents a group which may be converted into a carboxy group;
R₂ is a radical selected from H and pentanoyl.

2. The compound according to claim 1, wherein R₁ is COOR′, R′ being a radical selected from the group consisting of (C₁-C₆)-alkyl, substituted methyl, 2-substituted ethyl, 2,6-dialkyl-phenyl, benzyl, substituted benzyl and silyl,

3. The compound according to claim 2, wherein R′ is methyl.

4. The compound according to claim 2, wherein R′ is tert-butyl.

5. The compound according to claim 2, wherein R′ is benzyl.

6. The compound according to claim 1, wherein Y₁ and Y₂ are independently selected from the group consisting of: hydroxy, methoxy, ethoxy and phenoxy, or alternatively, Y₁ and Y₂ together with a boron atom form a cyclic structure selected from: 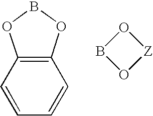
wherein Z is selected from the group consisting of (CH₂)ᵣCRᵤRᵥ(CH₂)ₛ and CRᵤRᵥ(CH₂)ₜCRᵤRᵥ; r and s are integers from 0 to 4 with the condition that r and s are not both 0; t is an integer from 0 to 1 and Rᵤ and Rᵥ are each independently selected from methyl and phenyl.

7. The compound according to claim 6, wherein Y₁ and Y₂ are hydroxy.

8. The compound according to claim 6, wherein Y₁ and Y₂ together with a boron atom form a cyclic structure, wherein Z is CH₂C(CH₃)₂CH₂.

9. The compound according to claim 6, wherein Y₁ and Y₂ together with a boron atom form a cyclic structure, wherein Z is C(CH₃)₂C(CH₃)₂.

10. The compound according to claim 1, which is selected from the group consisting of:
methyl N-(4-(5,5-dimethyl-[1,3,2]djoxaborinan-2-yl)phenyl-4-yl-methyl)-L-valinate;
methyl N-(4-(5,5-dimethyl-[1,3,2]dioxaborinan-2-yl)phenyl-4-yl-methyl N-pentanoyl-L-valinate;
methyl N-(4-(4,4,5,5-tetramethyl-[1,3,2]dioxaborolan-2-yl)phenyl-4-yl-methyl)-L-valinate; and
methyl N-(4-(4,4,5,5-tetramethyl-[1,3,2]dioxaborolan-2-yl)phenyl-4-yl-methyl)-N-pentanoyl-L-valinate.

11. The compound according to claim 1, which is selected from the group consisting of:
benzyl N-(4-(5,5-dimethyl-[1,3,2]dioxaborinan-2-yl)phenyl-4-yl-methyl)-L-valinate; and
benzyl N-(4-(5,5-dimethyl-[1,3,2]dioxaborinan-2-yl)phenyl-4-yl-methyl)-N-pentanoyl-L-valinate.

12. The compound according to claim 1, which is selected from the group consisting of:
tert-butyl N-(4-(5,5-dimethyl-[1,3,2]dioxaborinan-2-yl)phenyl-4-yl-methyl)-L-valinate; and
tert-butyl N-(4-(5,5-dimethyl-[1,3,2]dioxaborinan-2-yl)phenyl-4-yl-methyl)-N-pentanoyl-L-valinate.

13. The compound according to claim 1, which is selected from the group consisting of:
4-[(1-(S)-methoxycarbonyl-2-methyl-propylamino)-methyl]phenylboronic acid; and
4-{[(1-(S)-methoxycarbonyl-2-methyl-propyl)-pentanoyl-amino]-methyl}phenylboronic acid.

14. A preparation process of a compound of formula (II), as defined in claim 1, 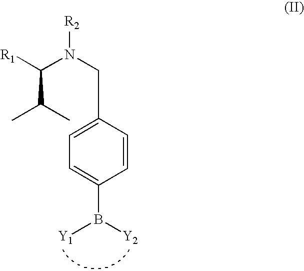
which comprises condensing a compound of formula (III) with a compound of formula (IV) or a salt thereof, followed by reducing the condensation product with an appropriate reducing agent, 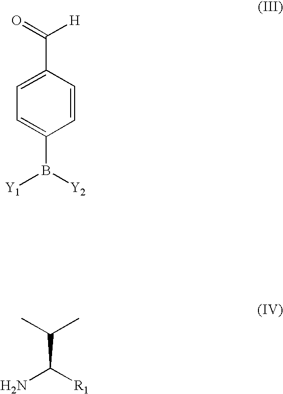
wherein R₁, R₂, Y_{1 and Y}₂ is a group as defined in claim 1, or alternatively, Y₁ and Y₂ is an intermediate form thereof which can be transformed to such a Y₁ and Y₂ groups; and thereafter, as necessary, transforming said intermediate forms of Y₁ and Y₂ groups to Y₁ and Y₂ groups as previously defined, and optionally submitting the compound obtained to an acylation reaction of the amino group with a pentanoyl halide.

15. The preparation process according to claim 14, wherein the condensation reaction is carried out in the presence of a base and a water scavenger in an appropriate solvent.

16. (canceled)

17. The preparation process according to claim 15, wherein the base is triethylamine.

18. (canceled)

19. (canceled)

20. The preparation process according to claim 14, wherein the reduction of the condensation product is carried out with a reducing agent selected from the group consisting of sodium borohydride and sodium cyanoborohydride.

21-24. (canceled)

25. A preparation process of Valsartan of formula (I) or a pharmaceutical salt thereof, 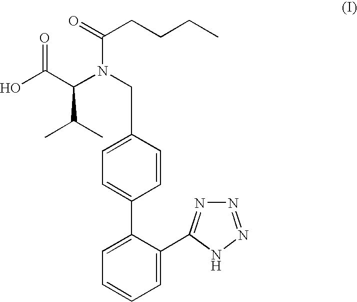
Which comprises the operations of:
a) coupling a compound of formula (II) as defined in claim 1, 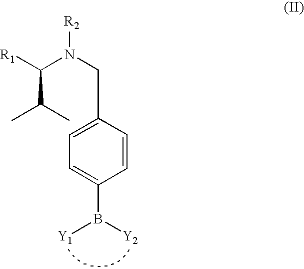
with a compound of formula (V), 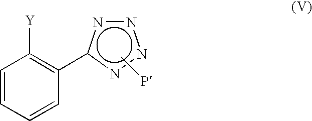
wherein Y is a leaving group and P′ is H or a protective group P; in an appropriate solvent system and in the presence of a metallic compound and a base, to give a compound of formula (I′); 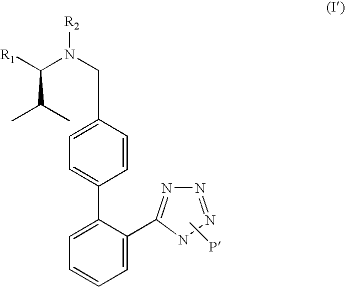
b) as necessary, submitting the compound obtained to a deprotection reaction to remove the protective group P′ and/or, as necessary, to an acylation reaction with a pentanoyl halide to introduce the pentanoyl moiety; and thereafter converting the compound of operation a) or the compound obtained in said operation b) into the free acid form of Valsartan or a salt thereof, by a hydrolysis, thermolysis, acidolysis or hydrogenolysis reaction; or alternatively, firstly submitting the compound of operation a) to a hydrolysis, thermolysis, acidolysis or hydrogenolysis reaction to yield the free acid form of valsartan or a salt thereof, or of an intermediate form of valsartan, and thereafter as necessary, submitting the compound obtained to a deprotection reaction to remove the protective group P′ and/or, as necessary, to an acylation reaction with a pentanoyl halide to introduce the pentanoyl moiety; and
c) if desired, converting the resulting free acid form of Valsartan into a salt thereof, or converting a resulting salt of Valsartan into the free acid form of Valsartan, or converting a resulting salt of Valsartan into a different salt.

26. The preparation process according to claim 25, where the compound obtained in operation a), firstly as necessary is submitted to a deprotection reaction to remove the protective group P′ and/or, as necessary, to an acylation reaction with a pentanoyl halide to introduce the pentanoyl moiety; and thereafter the compound obtained is converted into the free acid form of Valsartan or a salt thereof, by a hydrolysis, thermolysis, acidolysis or hydrogenolysis reaction.

27. The preparation process according to claim 25, where the compound obtained in operation a), firstly is submitted to a hydrolysis, thermolysis, acidolysis or hydrogenolysis reaction to yield the free acid form of valsartan or a salt thereof, or of an intermediate form of valsartan, and thereafter as necessary, submitting the compound obtained to a deprotection reaction to remove the protective group P′ and/or, as necessary, to an acylation reaction with a pentanoyl halide to introduce the pentanoyl moiety;

28. The preparation process according to claim 25 wherein the leaving group Y is Br.

29. The preparation process according to claim 25, wherein P′ is a protective group P.

30. (canceled)

31. The preparation process according to claim 25, wherein the metallic compound is selected from palladium, nickel, a metallic salt and a metallic complex.

32. The preparation process according to claim 31, wherein the metallic compound is a Pd complex, added or formed in situ, selected from the group consisting of PdX′₂, PdX′₂/PAr₃, PdX′₂/P(C₁-C₆)₃, PdX′₂/N(C₁-C₆)₃, PdL₄, and PdX′₂L₂; X′ is a leaving group independently selected from the group consisting of Cl, Br and OCOCH₃; Ar is an aromatic group selected from the group consisting of phenyl, tolyl and furyl; L is a ligand selected from the group consisting of NR′₃, SR′₂, and PR′₃; or alternatively in formula PdX′₂L₂ both L form a diphosphine of formula PR′₂—Z-PR′₂; R′ is independently selected from phenyl, tolyl, furyl, ferrocenyl and (C₁-C₆)-alkyl; and Z is selected from ferrocenyl and (C₁-C₄)-alkyl.

33. The preparation process according to claim 32, wherein the metallic compound is selected from the group consisting of tetrakis(triphenylphosphine)palladium (0), (Pd(PPh₃)₄); dichloro[1,1′-bis(diphenylphosphino)ferrocene]palladium (II), (PdCl₂(dppf)); 1,4-bis(diphenylphosphino)butane palladium (II) chloride, (PdCl₂(dppb)); dichlorobis(tricyclohexylphosphine) palladium (II), (PdCl₂(PCy₃)₂); dichloro[1,1′-bis(di-tert-butylphosphino)ferrocene]palladium (II), (PdCl₂(dtbp)); palladium black; palladium (II) chloride; palladium (II) acetate; mixtures of the previously mentioned catalysts with phosphines; and palladium catalysts over polymeric supports.

34. (canceled)

35. The preparation process according to claim 33, wherein the catalyst is PdCl₂(dppf).

36-39. (canceled)

40. The preparation process according to claim 25, wherein the base is selected from potassium carbonate and potassium phosphate.

41. The preparation process according to claim 25, wherein the protective group P is removed by submitting the compound obtained to a deprotection reaction with methanol, HCl/methanol or HCl/dioxane/water.

42. The preparation process according to claim 25, wherein when R₁ is COOR′ with R′=benzyl, p-methoxybenzyl or benzhydryl, the conversion of the compound of operation a) or b) into the free acid form of Valsartan or a salt thereof is carried out by hydrogenolysis in the presence of a palladium catalyst.

43. The preparation process according to claim 25, wherein when R₁ is COOR′ with R′=methyl, the conversion of the compound of operation a) or b) into the free acid form of Valsartan or a salt thereof is carried out by hydrolysis in suitable acid or basic conditions.
